(19) United States
(12) Patent Application Publication
(10) Pub. No.: US 2024/0082526 A1
(43) Pub. Date: Mar. 14, 2024

(54) MASK ASSEMBLY, HOLDING MEANS, VENTILATION APPARATUS, AND METHOD FOR PRODUCING A MASK ASSEMBLY

(71) Applicant: Drägerwerk AG & Co. KGaA, Lübeck (DE)

(72) Inventor: Hans-Ullrich HANSMANN, Lübeck (DE)

(21) Appl. No.: 18/263,177

(22) PCT Filed: Jan. 20, 2022
(86) PCT No.: PCT/DE2022/100057

§ 371 Date: Jul. 27, 2023

(30) Foreign Application Priority Data
Feb. 2, 2021 (DE) 10 2021 102 308.4

(51) Int. Cl. A61M 16/06

(52) U.S. Cl. CPC A61M 16/0683; A61M 16/0616; A61M 16/0666

(57) ABSTRACT

A mask device (10) for a ventilator (100) for ventilating a patient (11), has a breathing mask (12) for being positioned over the mouth (13) and over the nose (14) of the patient (11). A nasal cannula device (15) sends a ventilation gas into the nose (14) of the patient (11). A holding device (16) holds the nasal cannula device (15) at the breathing mask (12). The holding device (16) for the mask device (10), a ventilator (100) with the mask device (10) as well as a process for setting the mask device (10) are presented.

### CROSS REFERENCE TO RELATED APPLICATIONS

This application is a United States National Phase Application of International Application PCT/DE2022/100057, filed Jan. 20, 2022, and claims the benefit of priority under 35 U.S.C. § 119 of German Application 10 2021 102 308.4, filed Feb. 2, 2021, the entire contents of which are incorporated herein by reference.

### TECHNICAL FIELD

The present invention pertains to a mask device for a ventilator for ventilating a patient.

### BACKGROUND

Different ventilators with different breathing masks are known for so-called high-flow therapies in the state of the art. The prior-art devices are, as a rule, held in a relatively simple manner and cooperate directly with an oxygen source. Residues of exhaled air of the last breath of a patient are flushed out here and the next breath begins without the new tidal volume containing residues of the last breath. The percentage of carbon dioxide-containing, re-breathed air is thus greatly reduced. This technique helps patients who are able to produce work of breathing independently and sufficiently. A pressure assistance by the high-flow device is absent. The high-flow therapy is frequently used for patients who suffer under the efficiency of a pulmonary oxygen exchange. A typical device for this is a clinical blender or an oxygen flowmeter with nasal cannulas.

Furthermore, there are so-called CPAP devices, which provide a constant pressure, as a result of which the patient can breathe at this elevated pressure level. The airways are kept open and the gas exchange surface is increased by the increased pressure. The exhalation takes place usually via simple outflow openings. A continuous flushing takes place here as well. However, only the interior of the mask or a mask volume between the breathing mask and the patient is flushed. The mask volume is additive and is produced by the addition of the breathing mask. Typical clinical pictures for this are chronic obstructive pulmonary diseases (COPD) or acute respiratory distress syndrome (ARDS). The devices known for the treatment are used to flush the upper nasal airways with oxygen or with carbon dioxide-free room air.

Patients who are treated with high-flow devices have to be able to breathe reliably and sufficiently spontaneously. Based on the massive overflow technique employed, a monitoring of the breathing activity is hardly possible. In addition, a high gas consumption is possibly accepted. In addition to the costs and logistics for the gases, an additional humidifier is, as a rule, needed here as well. Irritations of the uppermost airways develop rapidly due to the high flow velocity. Patients who are treated with CPAP devices likewise must be able to breathe in a reliably spontaneous manner. However, the volume flows during the inhalation and during the exhalation differ greatly, so that a breathing pattern can be detected and monitored on the machine side. Flushing of the anatomical dead spaces does not take place.

### SUMMARY

An object of the present invention is to take the above-described problem at least partially into account. In particular, it is the object of the present invention to provide a system as well as a process for ventilating patients who require an elevated pressure level and at the same time a functional mass exchange, while the volume flow is kept as low as possible and a breathing pattern can be detected and/or monitored.

The above object is accomplished by features according to the invention. In particular, the above object is accomplished by the mask device having mask device features according to the invention, by the holding device having holding device features according to the invention, by the ventilator having ventilator features according to the invention as well as by the process having process features according to the invention. Further advantages of the present invention appear from the description and from the figures. Features that are described in connection with the mask device also apply in connection with the holding device according to the present invention, with the ventilator according to the present invention, with the process according to the present invention and also vice versa, so that reference is and/or can always be made concerning the disclosure to the individual aspects of the present invention.

According to a first aspect of the present invention, a mask device is provided for a ventilator for ventilating a patient. The mask device has a breathing mask to be positioned over the mouth and over the nose of the patient, a nasal cannula device for sending a breathing gas into the nose of the patient, and a holding device for holding the nasal cannula device at the breathing mask.

The solution according to the present invention is configured in terms of the device similarly to a CPAP device. However, the gas is not fed into the interior of the breathing mask, but directly into the nasal cannula device and from there into the nose of the patient or at least to a point located at a short distance in front of the nose of the patient. A desired pressure level can be established by the additional breathing mask. Accordingly, a breathing mask is proposed, which also makes it possible to feed gas into the interior of the mask or into a mask volume, but it feeds the gas mainly via the nose of the patient. The ventilation gas can again escape from the nasal space through the mouth as well as due to the leakage of the so-called prong of the nasal cannula device. However, the excess gas in the mask volume can now be maintained under a settable pressure. The pressure can be set by means of an overflow device or by means of an actively actuatable exhalation valve of the mask device. The gas flow can thus be used for flushing the upper nasal airways. The gas flow can, in addition, be used for a constant pressure assistance (CPAP) and for a varying pressure assistance (BIPAP).

A ventilation mode that maintains a continuous flushing flow can be set especially in connection with an active exhalation valve of the mask device. The ventilation pressure can then be set such that a fixed inhalation pressure is reached during the inhalation. An exhalation valve of the ventilator is predominantly closed in this state as long as the desired inhalation pressure is not exceeded substantially.

The exhalation valve can be regulated during the exhalation for setting the PEEP (Positive End Expiratory Pressure) to be maintained. At the same time, an inhalation valve of the ventilator is opened to the extent that fresh ventilation gas flows in the direction of the patient. Especially at the end of the exhalation, this ventilation gas ensures the flushing of the nasal dead spaces. Fresh, carbon dioxide-free ventilation gas is present in the nasal dead spaces before the beginning of the next inhalation.

The nasal cannula device has a hose for sending the ventilation gas through an opening in the breathing mask or past the breathing mask to the patient. At the end of the hose, the nasal cannula device preferably has a prong or a corresponding end piece or a gas discharge section for sending the ventilation gas into the nose of the patient. The holding device is preferably configured and embodied for fixing the hose at the breathing mask. The fact that the breathing mask is configured for being positioned over the mouth and the nose of the patient shall be defined especially such that the breathing mask is configured for covering the mouth and nose area of the patient in as fluid-tight a manner as possible, so that the mouth and the nose are located within the mask volume and are covered or enclosed by the breathing mask. A sealing effect can be created by the breathing mask between the area surrounding the breathing mask and the mask volume, i.e., the mask volume is sealed by the breathing mask against the area surrounding the breathing mask to the extent possible. A relatively high overall pressure level can be achieved hereby for the desired ventilation of the patient. A sealing effect can be created by the gas discharge section positioned at or in the nose between the mask volume and an inner volume of the nose. However, the sealing effect between the mask volume and the inner volume of the nose or a pharyngeal space adjoining same through the gas discharge section is preferably weaker than the sealing effect between the mask volume and the area surrounding the breathing mask. The gas discharge section may preferably be configured such that a certain leak is made deliberately possible between the inner volume of the nose and the mask volume. This can bring about an advantageous effect concerning the flushing of the upper airways and/or of the pharyngeal space.

According to another embodiment of the present invention, it is possible that the breathing mask in a mask device has a mask wall with a passage opening and the nasal cannula device has a hose, which extends through the passage opening for sending the ventilation gas to the patient. The mask device can thus be provided in a compact form. Due to the hose being passed through the breathing mask or through the mask wall, the desired fluid tightness of the mask device can be achieved in a relatively reliable manner, for example, compared to a hose led past the breathing mask. The hose correspondingly extends in such an embodiment from an area located outside of the breathing mask through the mask wall in the direction of the nose of the patient using the mask device.

Furthermore, it is possible that the holding device in a mask device has a fastening device for fixing the hose in a holding position at the breathing mask as well as for releasing the hose from the holding position for a displacement motion between the hose and the breathing mask. This means that the fastening device can be adjusted into at least two different functional states in order to make possible the displacement of the hose at the breathing mask, on the one hand, and, on the other hand, to fix the hose at the breathing mask as soon as the hose is in the desired position at the breathing mask and as soon as the end of the hose and/or the prong are located at the desired position at or in the nose of the patient. The fastening device is configured for fixing at least one part of the hose in and/or at the breathing mask or in a fixed position relative to the breathing mask and/or to the opening of the mask. This means that the hose does not need to be fastened or to have been fastened directly to the breathing mask or adjoining the breathing mask. The fastening device may rather be considered to be a binding member for the fastening between the hose and the breathing mask.

Further, it may be advantageous if the fastening device in a mask device according to the present invention closes a passage area between an outer side of the hose and an inner circumferential surface of the passage opening during the fixation of the hose in the holding position. The fastening device may consequently be used as a sealing device for closing the passage opening in a fluid-tight manner or for sealing the mask volume against the surrounding area of the mask device.

It is, moreover, possible in a mask device according to the present invention that at least a part of the fastening device is fastened to the mask wall and/or to the hose. A stable connection of the components can thus be established between the breathing mask and the hose in a simple and compact manner.

According to another embodiment variant of the present invention, it is possible that in a mask device, the fastening device has at the mask wall a first passage sleeve with an internal thread tapering conically towards the patient wearing the breathing mask and a screw with a conical external thread, which screw is arranged in a jacket-like manner (jacketing) around a part of the hose for screwing to the conical internal thread of the first passage sleeve and thereby for fixing the hose in the holding position. This provides an effective fastening device in a simple and compact manner for the flexible fastening of the hose to the breathing mask or in the passage opening. The screw may be elastically deformed by screwing to the passage sleeve, so that the screw will be fixed to the hose by the screwing to the passage sleeve. The screw may have the elasticity due to corresponding properties of the material, due to a suitable dimensioning and/or due to a gap extending over the entire length of the screw in the jacket-like (jacket) screw wall to reduce the screw diameter during the screwing.

Further, it is possible that the fastening device in a mask device has at the mask wall an elastically deformable second passage sleeve configured in a jacket-like manner around a part of the hose with an external thread, especially with an at least partially conical external thread, and a nut with a conical internal thread for being screwed to the external thread of the second passage sleeve and thereby for fixing the hose in the holding position.

Moreover, it is possible in a mask device according to the present invention that, when viewed in the radial direction, an elastically deformable clamping sleeve is formed in a jacket-like manner around a part of the hose between a hose outer side and an inner circumferential surface of the second passage sleeve, the material of the clamping sleeve having a higher strength than the material of the hose. The hose can thus be protected from being damaged by the clamping sleeve. In addition, a better distribution of forces on the hose can be achieved thereby. The clamping sleeve and/or the material of the clamping sleeve preferably have a higher strength than the hose, than the material of the hose and/or than the hose in the area in which the clamping sleeve is formed in a jacket-like manner around the hose. The clamping sleeve can be positioned at the hose loosely and/or displaceably in the released state. The threads are all configured in the above-described embodiments for screw connection outside of the breathing mask and outside of the mask volume. Good operability of the fastening device can thus be guaranteed.

Furthermore, it is possible that in a mask device according to the present invention, the fastening device has an elastically deformable diaphragm suction sleeve arranged at the mask wall with a diaphragm inner volume and the diaphragm suction sleeve is configured in a jacket-like manner around the hose in the area of the passage opening, wherein the diaphragm suction sleeve has a pressure port for generating a vacuum in the diaphragm inner volume. The desired manner of functioning of the fastening device can be achieved hereby as well. The diaphragm suction sleeve is preferably fastened to the breathing mask or to the mask wall in the area of the passage opening. A pump can be connected to the pressure port to generate the vacuum. The vacuum can accordingly be generated by means of the pressure port, i.e., not alone by the pressure port. Air or a gas, which is located in the diaphragm inner volume, can be suctioned from the diaphragm inner volume via the pressure port. The diaphragm inner volume decreases thereby and the diaphragm suction sleeve fixes the hose in the passage opening. The diaphragm suction sleeve may be configured in a ring-shaped manner (ring shape) around the hose in the area of the passage opening. The diaphragm suction sleeve may further be fastened to the mask wall in a positive-locking connection, a non-positive connection or by a connection in substance. It is further possible in such a mask device that an elastically and/or plastically deformable solid is arranged in the diaphragm inner volume. The hose can thus be fixed in the desired position in an especially stable manner.

In addition, the fastening device in a mask device according to the present invention may have in the area of the passage opening an elastically deformable diaphragm pressure sleeve with a diaphragm inner volume, which diaphragm pressure sleeve is arranged in a jacket-like manner and/or in a ring-shaped manner around the hose, wherein the diaphragm pressure sleeve has a pressure port for generating an overpressure in the diaphragm inner volume. The diaphragm pressure sleeve on the hose can thus be pushed into the passage opening, the hose can be moved into the desired position and the diaphragm pressure sleeve can subsequently be inflated, so that this will be fixed in the passage opening between the hose and the mask wall and it will thus hold the hose in the desired position at the breathing mask.

In another embodiment of the mask device, the hose may have a thinner hose wall in the area of the passage opening than in an area outside of the passage opening. As an alternative or in addition, the hose may additionally have in a mask device according to the present invention an elastically deformable hose wall and/or a hose wall with a higher elasticity in the area of the passage opening than in an area outside of the passage opening. As soon as ventilation gas is sent through the hose, the hose can expand in the area of the thinner and/or elastic or more elastic hose wall due to the overpressure generated in the hose or increase in the radial direction and fix the hose in the passage opening. The area of the thinner and/or more flexible hose wall preferably extends over an area corresponding to several times the wall thickness of the mask wall, so that the hose can be fixed in the desired position in a sufficiently flexible manner. The thinner and/or more flexible hose wall preferably extends over a length in a range of 5 mm to 30 mm. The thinner and/or more flexible hose wall may be configured as a monolithic component of the hose.

According to another embodiment variant of the present invention, it is possible that the fastening device in a mask device has a thermoplastically deformable connection jacket, which is formed in a jacket-like manner around the hose and is arranged at the mask wall in the area of the passage opening. The thermoplastic material of the fastening device is configured such that it is relatively firm at the usual ambient temperature, so that the fastening device is in a holding state for fixing the hose. If the hose shall be released from the holding position for the displacement motion, the connection jacket must only be heated.

It may, furthermore, be advantageous in the above-described embodiments if the mask device has a connection hinge for the articulated holding of the hose in the passage opening. It can thus be ensured that the hose will not slide out of the passage opening completely or too far as soon as the hose is not fastened by the fastening device at and/or in the breathing mask. However, the hose can nevertheless still be displaced by the connection hinge in the area of the passage opening for the desired positioning. The connection hinge may be formed within and/or outside of the passage opening at the breathing mask as well as at the hose.

In a mask device according to the present invention, a part of the hose, which is located during the use of the mask device in a mask volume formed by the breathing mask at the patient, may be plastically deformable. The position of the hose end or the position of a gas discharge section at and/or in the nose can be adapted to the particular patient or be brought into the desired position in a flexible manner thereby as well.

In addition, it is possible that the nasal cannula device in a mask device according to the present invention has a gas discharge for discharging the ventilation gas into the nose of the patient, the gas discharge having a nozzle-type (nozzle) configuration. A targeted gas discharge, which can send the ventilation gas via the gas discharge into the desired direction, can thus be generated. It is thus possible to position the nasal cannulas or the prong at a short distance or just barely in front of the nose of the patient rather than in the nose. This contributes to an improved wear comfort and consequently to better acceptance of the mask device.

It may also be advantageous if the nasal cannula device of a mask device according to the present invention has a gas discharge section for discharging the ventilation gas into the nose of the patient, in which case the gas discharge section tapers conically in a ventilation gas flow direction. The gas discharge section of the nasal cannula device can thus be positioned in the nose of the patient in a simple and reliable manner.

Furthermore, the nasal cannula device of a mask device according to the present invention may have a gas discharge section for discharging the ventilation gas into the nose of the patient, wherein a balloon seal with a balloon jacket is configured in and/or at the gas discharge section and wherein the balloon jacket has a gas opening for feeding the ventilation gas in the nasal cannula device during a ventilation of the patient into a balloon volume formed by the balloon jacket for holding the gas discharge section in the nose in a pressurized manner. Stable positioning of the gas discharge section in the nose of the patient can thus be achieved.

Moreover, the nasal cannula device of a mask device may have a gas discharge section for discharging the ventilation gas into the nose of the patient, wherein the mask device further has an inflatable hose seal, which is formed around the gas discharge section in a ring-shaped manner. This is also useful for positioning the gas discharge section in the nose of the patient in a stable manner with a limited motion. The hose seal may have a pressure port, via which the hose seal can be inflated by means of a pump and can thus be fixed in the nose of the patient. A compressed air line for inflating the hose seal may be led outside of the breathing mask into the hose and extend there to the hose seal and/or to the above-mentioned pressure port.

According to another embodiment of the present invention, it is possible that the nasal cannula device has a gas discharge section for discharging the ventilation gas into the nose of the patient, wherein the gas discharge section has at least one elastically deformable spring-loaded arm for holding the gas discharge section in the nose in a pressurized manner. The at least one spring-loaded arm is a simple and yet reliable possibility for a stable positioning of the gas discharge section in the nose of the patient. The at least one spring-loaded arm may, moreover, be configured such as to ensure that gas can continue to flow out of the nose of the patient. The gas discharge section preferably has at least two or four spring-loaded arms. Instead of the spring-loaded arms, spring funnels may also be formed at the gas discharge section. These lead to an even more stable positioning of the gas discharge section in the nose of the patient. In addition, these prevent a discharge of gas from the nose of the patient at least to a very great extent.

It may also be advantageous if a mask device according to the present invention has a holding strap device with a headband and/or with a neckband at the breathing mask for holding the breathing mask at the patient. Not only the breathing mask, but also the nasal cannula device can thus be held in the desired position in a stable manner. Furthermore, the headband and/or the neckband may have an adjustable configuration for adapting the mask device to the particular patient. During the use of the mask device, the breathing mask must be pressed or pulled onto the face of the patient by the overpressure without leaks to the extent possible. The leaks can be maintained with the strapping according to the present invention at values below 10 L/min or below 20 L/min. The neckband can be defined as a strapping for holding the breathing mask in the area of the corner of the mouth of the patient. The headband can be defined as a strapping for holding the breathing mask in the area of the root of the nose of the patient. The lengths of both straps can be set and adjusted by means of a Velcro fastener. The elasticity of the straps is as low as possible and/or the straps have a correspondingly rigid configuration or are configured with a correspondingly high strength.

The breathing mask of a mask device according to the present invention may have a mask edge for placing the breathing mask on the face of the patient, the edge of the mask having an all-round, elastically deformable silicone lip and/or a correspondingly shaped elastomer. It is thus possible to bring about a reduced leak from the mask volume into the area surrounding the mask device along with high wear comfort of the breathing mask.

Furthermore, it is possible in the case of a mask device according to the present invention for the breathing mask to have an elastically deformable mask edge for placing the breathing mask on the face of the patient, wherein the mask edge has an edge chamber with an edge volume, in which overpressure is present relative to the area surrounding the mask device. A high level of wear comfort can be created hereby as well along with an effective sealing effect at the same time.

For a high level of wear comfort and for the desired sealing effect, the breathing mask of a mask device according to the present invention may have, in addition, a gel-like and/or sponge-like mask edge for placing the breathing mask on the face of the patient.

According to another aspect of the present invention, a holding device is provided for holding a nasal cannula device at the breathing mask in a mask device as described above. In addition, a ventilator, which has a mask device as described above, is provided for ventilating a patient. The holding device according to the present invention as well as the ventilator according to the present invention bring with them the same advantages as those described in detail in reference to the mask device according to the present invention.

Another aspect of the present invention pertains to a process for setting a mask device as described above, wherein the nasal cannula device has a hose for sending the ventilation gas and a gas discharge section for discharging the ventilation gas into the nose of the patient. The holding device comprises, for a displacement motion between the hose of the breathing mask, a fastening device for fixing the hose in a holding position at the breathing mask as well as for releasing the hose from the holding position. The process has the following steps:
positioning of the breathing mask over the mouth and over the nose of the patient,
displacement of the hose relative to the breathing mask for positioning the gas discharge section in or at the nose of the patient, and
fixing of the hose by the fastening device in the holding position.

Thus, the process according to the present invention brings with it the above-described advantages as well. In particular, the device features described above in connection with the holding device as well as with the fastening device shall be considered to be corresponding process features within the framework of a process according to the present invention for fixing the hose in the holding position and for releasing the hose from the holding position.

Further steps improving the present invention appear from the following description of different exemplary embodiments of the present invention, which are shown schematically in the figures. All the features and/or advantages appearing from the claims, from the description or from the figures, including structural details and arrangements in space, may be essential for the present invention both in themselves and in the different combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a ventilator with a mask device according to a first embodiment of the present invention;

FIG. 2 is a schematic view showing details of a mask device according to a second embodiment of the present invention;

FIG. 3 is a schematic view showing details of a mask device according to a third embodiment of the present invention in a first functional state;

FIG. 4 is a schematic view showing details of a mask device according to the third embodiment of the present invention in a second functional state;

FIG. 5 is a schematic view showing details of a mask device according to a fourth embodiment of the present invention in a first functional state;

FIG. 6 is a schematic view showing details of a mask device according to the fourth embodiment of the present invention in a second functional state,

FIG. 7 is a schematic view showing details of a mask device according to a fifth embodiment of the present invention in a first functional state;

FIG. 8 is a schematic view showing details of a mask device according to the fifth embodiment of the present invention in a second functional state;

FIG. 9 is a schematic view showing details of a mask device according to a sixth embodiment of the present invention;

FIG. 10 is a schematic view showing details of a mask device according to a seventh embodiment of the present invention;

FIG. 11 is a schematic view showing details of a mask device according to an eighth embodiment of the present invention;

FIG. 12 is a schematic view showing details of a mask device according to a ninth embodiment of the present invention in a first functional state;

FIG. 13 is a schematic view showing details of a mask device according to the ninth embodiment of the present invention in a second functional state;

FIG. 14 is a schematic view showing details of a mask device according to a tenth embodiment of the present invention in a first functional state;

FIG. 15 is a schematic view showing details of a mask device according to the tenth embodiment of the present invention in a second functional state;

FIG. 16 is a schematic view showing details of a mask device according to an eleventh embodiment of the present invention;

FIG. 17 is a schematic view showing details of a mask device according to a twelfth embodiment of the present invention;

FIG. 18 is a schematic view showing details of a mask device according to a thirteenth embodiment of the present invention;

FIG. 19 is a schematic view showing details of a mask device according to a fourteenth embodiment of the present invention;

FIG. 20 is a schematic view showing details of a mask device according to a fifteenth embodiment of the present invention;

FIG. 21 is a schematic view showing details of a mask device according to a sixteenth embodiment of the present invention;

FIG. 22 is a schematic view showing details of a mask device according to a seventeenth embodiment of the present invention; and

FIG. 23 is a flow chart for explaining a process according to the present invention.

### DESCRIPTION OF PREFERRED EMBODIMENTS

Referring to the drawings, elements having the same function and mode of operation are always provided with the same reference numbers in the figures.

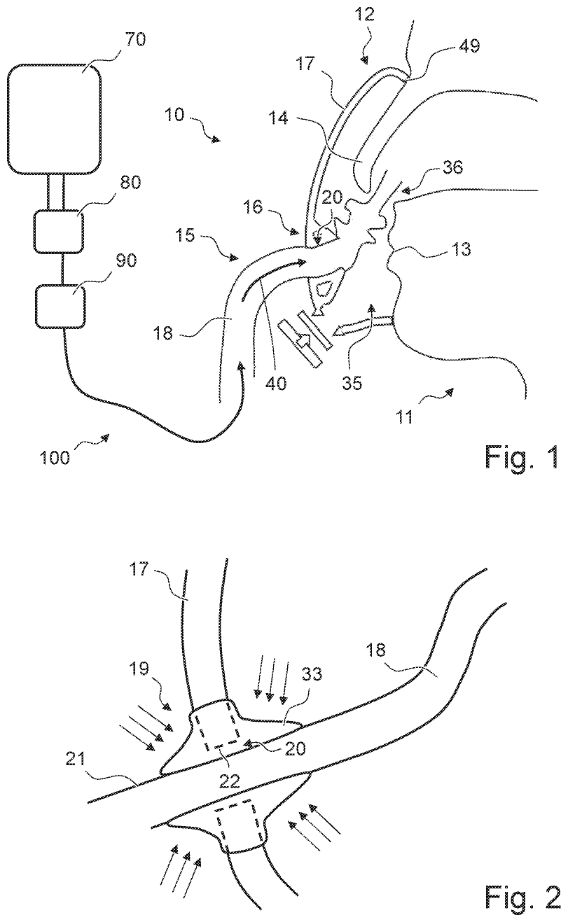

FIG. 1 shows a ventilator 100 for ventilating a patient 11. The ventilator 100 has a gas source 70, a blender 80, a humidifier 90 and a mask device 10. The gas source 70 has two pressure ports for oxygen and for medical air. The two gas streams are merged in the blender 80. The set mixture of air and oxygen is sent from there with a possibly constant volume flow via the humidifier 90 as a ventilation gas in a ventilation direction 40 to the patient 11.

The mask device 10 comprises a breathing mask 12, which is positioned over or on the mouth 13 and the nose 14 of the patient 11. Further, the mask device 10 comprises a nasal cannula device 15 for sending the ventilation gas into the nose 14 of the patient 11 and a holding device 16 for holding the nasal cannula device 15 at the breathing mask 12. The breathing mask 12 has a mask wall 17 with a passage opening 20. The nasal cannula device 15 has a hose 18, which extends through the passage opening 20 for sending the ventilation gas to the patient 11. To adapt the nasal cannula device 15 to the particular patient 11, an end section of the hose 18 is plastically deformable in the mask volume 35. The hose 18 has a gas discharge section 36 in the area of the nose 14 of the patient 11.

A sealing effect is created by the breathing mask 12 between the area surrounding the breathing mask 12 and the mask volume 35. This means that the mask volume 35 is sealed by the breathing mask 12 against the area surrounding the breathing mask to the extent possible. As a result, a relatively high overall pressure level is reached for the desired ventilation of the patient 11. A sealing effect can be generated between the mask volume 35 and an internal volume of the nose by the gas discharge section 36 positioned at or in the nose 14. The sealing effect between the mask volume 35 and the internal volume of the nose through the gas discharge section 36 is, however, weaker than the sealing effect between the mask volume 35 and the area surrounding the breathing mask 12. The gas discharge section 36 is therefore configured in the embodiment shown such that a certain leakage is made possible in the area of the gas discharge section 36 between the internal volume of the nose and the mask volume 35.

FIG. 2 shows a detail view of a solution of a mask device 10 according to a second embodiment. According to the embodiment shown in FIG. 2, the holding device 16 comprises a fastening device 19 for fixing the hose 18 in a holding position at the breathing mask 12 or at and in the passage opening 20 as well as for releasing the hose 18 from the holding position for a displacement motion between the hose 18 and the breathing mask 12. The fastening device 19 shown in FIG. 2 closes a passage area between an outer side 21 of the hose 18 and an inner circumferential surface 22 of the passage opening 20 when the hose 18 is fixed in the holding position. The fastening device 19 shown has a thermoplastically deformable connection jacket 33, which is formed in a jacket-like manner around the hose 18 and which is arranged at the mask wall 17 in the area of the passage opening 20. If the connection jacket 33 is heated, the hose 18 can move in the passage opening 20 and in the connection jacket 33 and can be brought into the desired position. If the connection jacket 33 cools again or assumes ambient temperature, the hose 18 is fixed in its position. The connection jacket 33 is fastened in the area of the passage opening 20 to the mask wall 17.

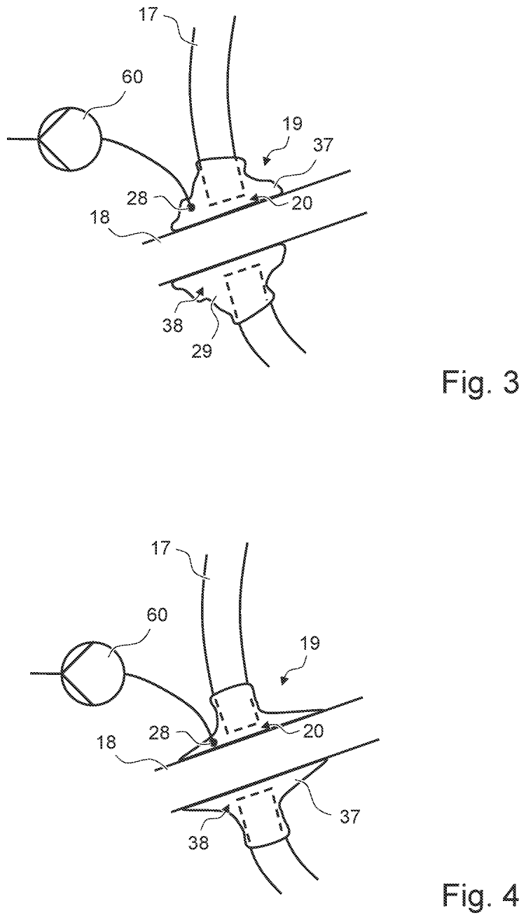

FIG. 3 shows a detail view of a solution of a mask device 10 according to a third embodiment. According to the embodiment shown in FIG. 3, the fastening device 19 comprises an elastically deformable diaphragm suction sleeve 37 arranged at the mask wall 17 with a diaphragm inner volume 38, wherein the diaphragm suction sleeve 37 is configured in a jacket-like manner around the hose 18 in the area of the passage opening 20 and wherein the diaphragm suction sleeve 37 has a pressure port 28 for generating a vacuum in the diaphragm inner volume 38. Further, an elastically deformable solid 29 in the form of Styrofoam is arranged in the diaphragm inner volume 38. FIG. 3 shows the diaphragm suction sleeve 37 in a normal pressure state. FIG. 4 shows the diaphragm suction sleeve 37 in a vacuum state, in which vacuum was generated or has been generated in the diaphragm suction sleeve 37 by a pump 60 and the diaphragm suction sleeve 37 is attached as a result by suction to the hose 18 and thus fastens this to the breathing mask 12.

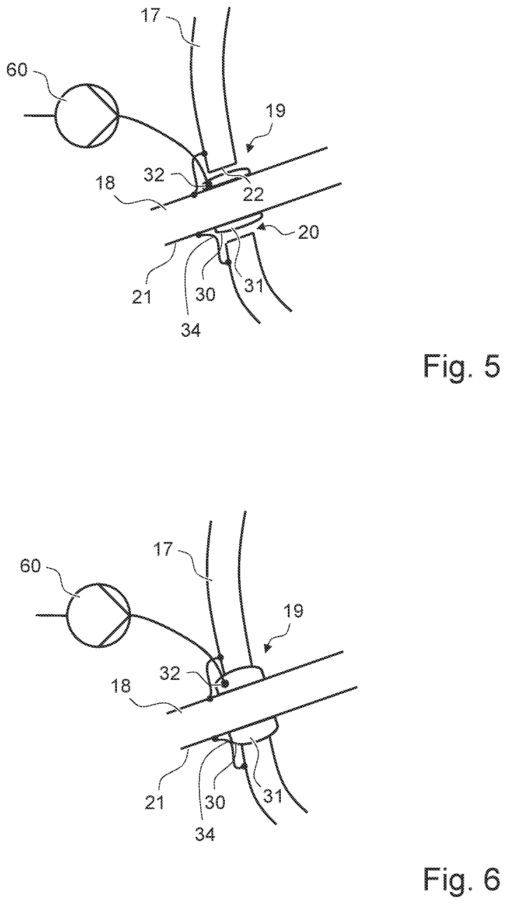

FIG. 5 shows a detail view of a solution of a mask device 10 according to a fourth embodiment. According to the embodiment shown in FIG. 5, the fastening device 19 has in the area of the passage opening 20 an elastically deformable diaphragm pressure sleeve 30 arranged in a jacket-like manner around the hose 18 with a diaphragm inner volume 31. The diaphragm pressure sleeve 30 has a pressure port 32 for generating an overpressure in the diaphragm inner volume 30. Further, the mask device 10 shown in FIG. 5 has a connection hinge 34 in the form of two flexible connection straps for holding the hose 18 in an articulated manner in the passage opening 20. FIG. 5 shows the diaphragm pressure sleeve 30 in a normal pressure state, in which the hose 18 can be moved or displaced in the passage opening 20 for the desired positioning. FIG. 6 shows the diaphragm pressure sleeve 30 in an overpressure state, in which overpressure was generated or has been generated by a pump 60 in the diaphragm pressure sleeve 30 and the diaphragm pressure sleeve 30 expands or is inflated thereby in the passage opening 20 until the hose 18 is fixed in the desired position at the breathing mask 12.

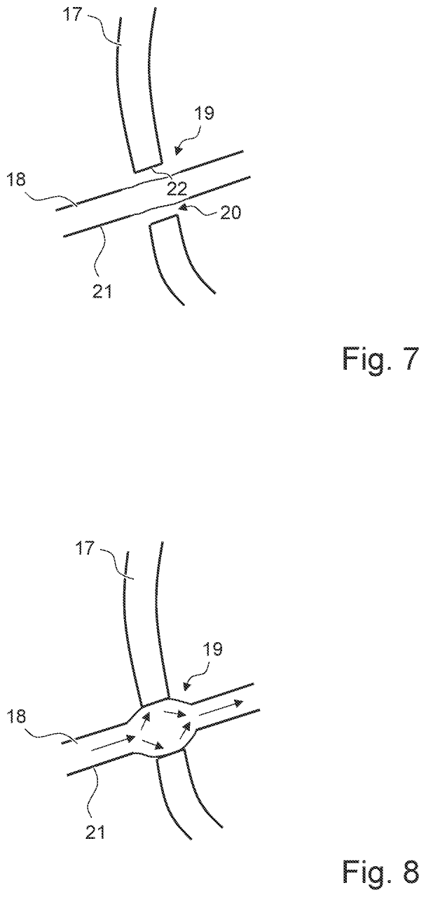

FIG. 7 shows a detail view of a solution of a mask device 10 according to a fifth embodiment. According to the embodiment shown in FIG. 7, the hose 18 has, in the area of the passage opening 20, a thinner hose wall than in an area outside of the passage opening 20. The hose 18 thus has a higher elasticity in the area of the passage opening 20 than in the area outside of the passage opening 20. If, as is shown in FIG. 8, ventilation gas is sent through the hose 18 to the patient 11, the elastic or more elastic hose wall expands in the area of the passage opening 20 due to the increased gas pressure in the hose 18 radially outwards and thus fixes the hose 18 in the passage opening 20.

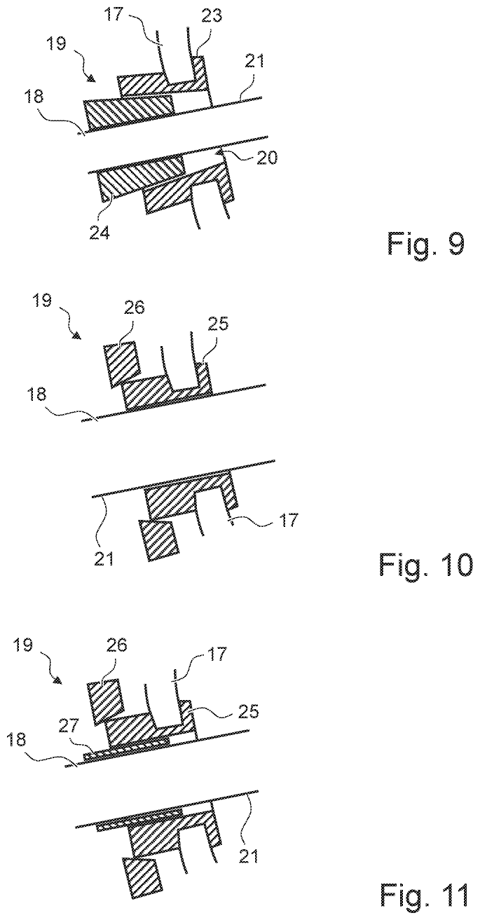

FIG. 9 shows a detail view of a solution of a mask device 10 according to a sixth embodiment. According to the embodiment shown in FIG. 9, the fastening device 19 has at the mask wall 17 a first passage sleeve 23 with an internal thread tapering conically towards the patient 11, who is wearing the breathing mask 12, and a screw 24 or screw sleeve arranged in a jacket-like manner around a part of the hose 18 with a conical external thread for screwing to the conical internal thread of the first passage sleeve 23 and thereby for fixing the hose 18 in the holding position.

According to the seventh embodiment shown in FIG. 10, the fastening device 19 has at the mask wall 17 an elastically deformable, second passage sleeve 25 formed in a jacket-like manner around a part of the hose 18 with an external thread and a nut 26 with a conical internal thread for screwing to the external thread of the second passage sleeve 25 and thereby for fixing the hose 18 in the holding position.

FIG. 11 shows a detail view of a solution of a mask device 10 according to an eighth embodiment, in which, when viewed in the radial direction, an elastically deformable clamping sleeve 27 is formed in a jacket-like manner around a part of the hose 18 between a hose outer side 21 and an inner circumferential surface of the second passage sleeve 25. The material of the clamping sleeve 27 has a higher strength here than the material of the hose 18.

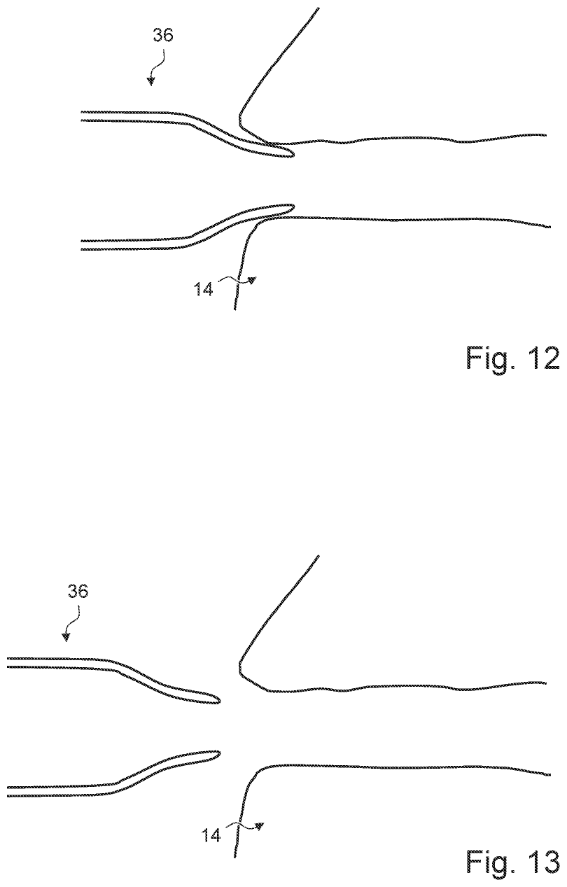

FIG. 12 shows a detail view of a solution of a mask device 10 according to a ninth embodiment. Here, the nasal cannula device 15 has a nozzle-type gas discharge 36 for discharging the ventilation gas into the nose 14 or to the nose of the patient 11. As is shown in FIG. 13, the gas discharge 36 or the end section of a prong can thus be positioned outside of the nose 14 without a major impairment of the efficiency of the ventilation gas supply.

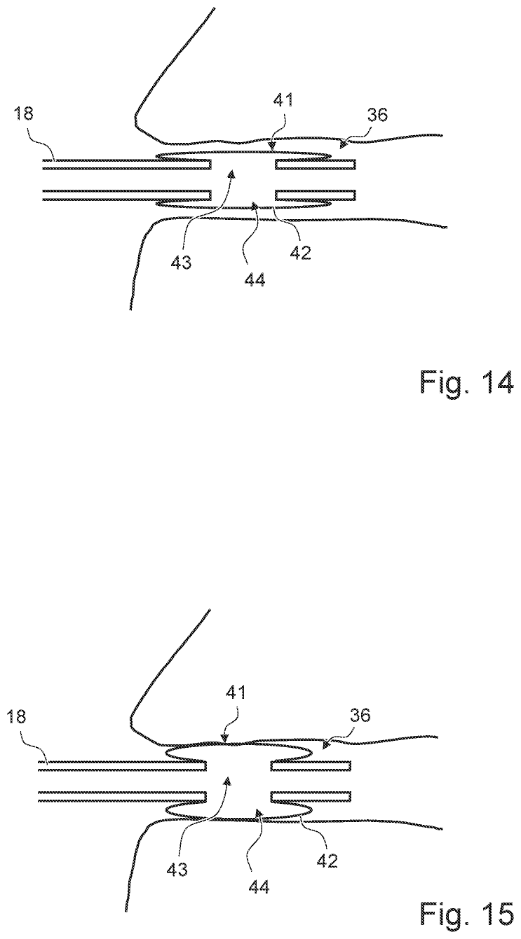

FIG. 14 shows a detail view of a solution of a mask device 10 according to a tenth embodiment, in which the nasal cannula device 15 has a gas discharge section 36 for discharging the ventilation gas into the nose 14 of the patient 11, wherein a balloon seal 41 with a balloon jacket 42 is formed at the gas discharge section 36 and wherein the balloon jacket 42 has a gas opening 43 for feeding the ventilation gas in the nasal cannula device 15 during a ventilation of the patient 11 into a balloon volume 44 formed by the balloon jacket 42 for holding the gas discharge section 36 in the nose 14 in a pressurized manner. As is shown in FIG. 15, the balloon volume 44 is filled automatically with the ventilation gas during the ventilation of the patient 11 and it expands as a result. The pressure in the balloon volume is just high enough for holding the gas discharge section 36 in the nose 14 in a stable manner and for not generating thereby an unpleasant pressure acting on the wall of the nose.

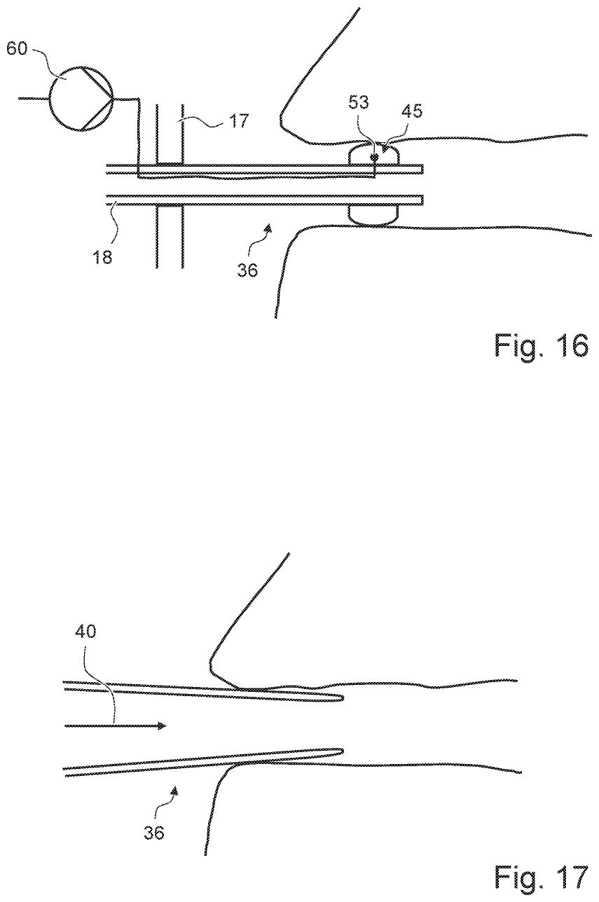

FIG. 16 shows a detail view of a solution of a mask device 10 according to an eleventh embodiment, in which the mask device 10 has an inflatable hose seal 45, which is formed in a ring-shaped manner around the gas discharge section 36. The hose seal 45 shown has a pressure port 53, via which the hose seal 45 can be inflated by means of a pump 60 and thus can be fixed in the nose of the patient 11 such that there is only a limited motion. A compressed air line 54 for inflating the hose seal 45 is led into the hose 18 on the outside of the breathing mask 12 and it extends there within the hose 18 up to the hose seal 45 or to the pressure port 53.

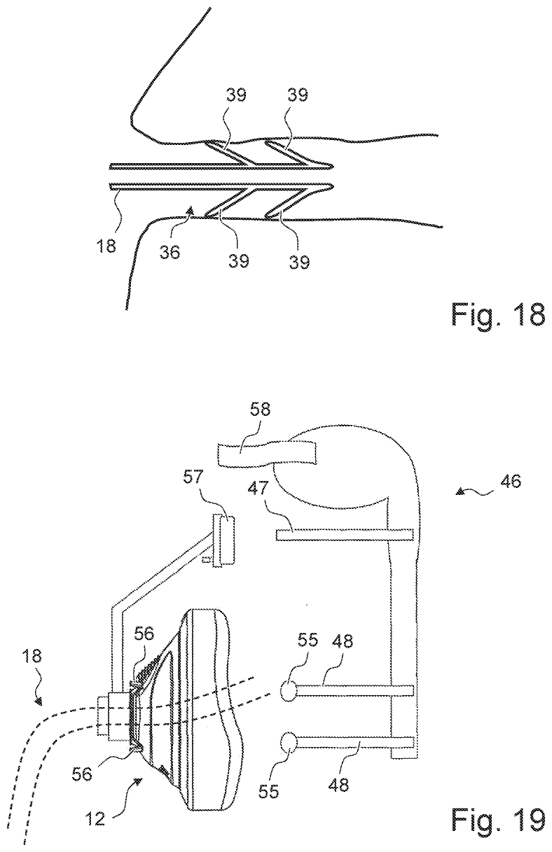

FIG. 17 shows a detail view of a solution of a mask device 10 according to a twelfth embodiment, in which the gas discharge section 36 is tapered conically in a ventilation gas flow direction 40. In the detail shown of a thirteenth embodiment of the mask device 10, which is shown in FIG. 18, the gas discharge section 36 has two elastically deformable spring-loaded arms 39 for holding the gas discharge section 36 in the nose 14 in a pressurized and tensioned manner.

FIG. 19 shows further details of solutions for a mask device 10 according to a fourteenth embodiment. As is shown in FIG. 19, the mask device 10 has a holding strap device 46 with a headband 47, with an upper headband 58 and two neckbands 48 for holding the breathing mask 12 at the patient 11. Holding rings 55, which can be fastened to corresponding hooks 56 for holding the breathing mask 12, are fastened to the neckbands 48. The headband 47 and the headband 58 may be attached to a holding eye 57. As an alternative to the strapping shown, it is also possible to use straps with Velcro fastener instead of the holding rings 55 and hooks 56. The headband 58 may be omitted. In addition, one of the neckbands 48 may be omitted.

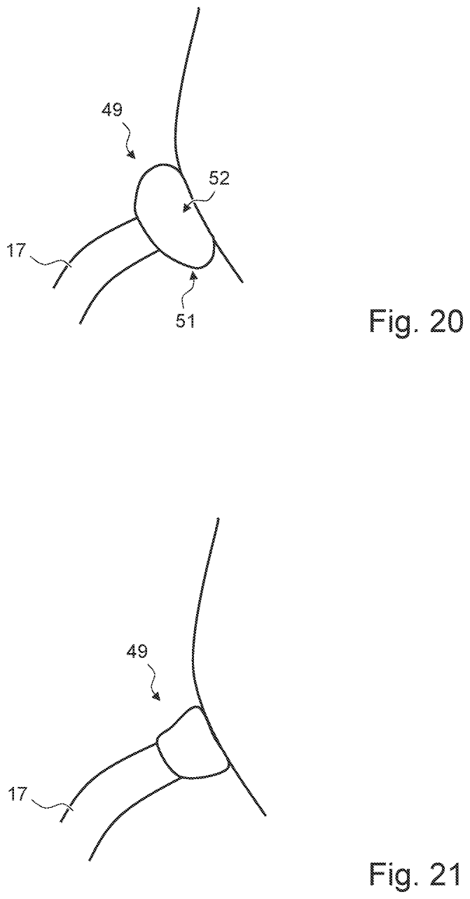

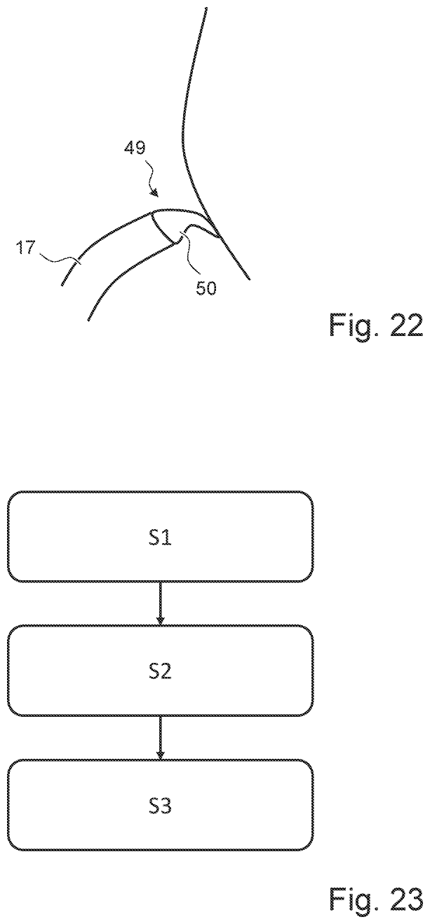

Possible embodiment variants of a mask edge 49 of the breathing mask 12 will be described with reference to FIGS. 20 through 22. FIG. 20 shows a mask device 10 according to a fifteenth embodiment, in which the breathing mask 12 has an elastically deformable mask edge 49 for being placed over the face of the patient 11, wherein the mask edge 49 has an edge chamber 51 with an edge volume 52, in which overpressure prevails relative to the area surrounding the mask device 10. FIG. 21 shows a detail solution of a sixteenth embodiment of the mask device 10, in which the breathing mask 12 has a gel-like mask edge 49 for placing the breathing mask 12 over the face of the patient 11. The mask edge 49 of a mask device 10 according to a seventeenth embodiment, which [mask edge] is shown in FIG. 22, has an all-round, elastically deformable silicone lip 50.

With reference to FIG. 23, a process for setting a mask device 10 as described above will subsequently be described. In a first step S1, the breathing mask 12 is positioned first over the mouth 13 and over the nose 14 of the patient 11. In a second, especially subsequent step S2, the hose 18 is displaced relative to the breathing mask 12 in order to bring the gas discharge section 36 into the desired position in or at the nose 14 of the patient 11. The hose 18 is fixed by the fastening device 19 in the holding position in a third step S3. The ventilation of the patient 1 can then be started.

The present invention may have other configuration principles in addition to the embodiments described. In other words, the present invention shall not be considered to be limited to the exemplary embodiments explained in reference to the figures. Only one nostril and only one gas discharge section 36 are shown in FIGS. 12 through 18. The gas discharge sections 36 shown are, however, present, as a rule, as two gas discharge sections in a mask device 10 according to the present invention, for example, at a correspondingly configured prong.

While specific embodiments of the invention have been shown and described in detail to illustrate the application of the principles of the invention, it will be understood that the invention may be embodied otherwise without departing from such principles.

1. A mask device for a ventilator for ventilating a patient, the mask device comprising:
a breathing mask configured to be positioned over the mouth and over the nose of the patient,
a nasal cannula device for sending a ventilation gas into the nose of the patient; and
a holding device holding the nasal cannula device at the breathing mask.

2. A mask device in accordance with claim 1, wherein the breathing mask comprises a mask wall with a passage opening and the nasal cannula device comprises a hose, which extends to the patient through the passage opening for sending the ventilation gas to the patient.

3. A mask device in accordance with claim 1, wherein the holding device comprises a fastening device configured to fix the hose in a holding position at the breathing mask as well as to release the hose from the holding position, for a displacement motion between the hose and the breathing mask.

4. A mask device in accordance with claim 3, wherein the fastening device closes a passage area between a hose outer side of the hose and an inner circumferential surface of the passage opening in a fixed state of the hose in the holding position.

5. A mask device in accordance with claim 3, wherein at least a part of the fastening device is fastened to the mask wall and/or to the hose.

6. A mask device in accordance with claim 3, wherein the fastening device comprises a first passage sleeve with an internal thread tapering conically towards the patient, who wears the breathing mask, at the mask wall, and a screw arranged jacketing around a part of the hose at the hose with a conical external thread for being screwed to the conical internal thread of the first passage sleeve and thereby for fixing the hose in the holding position.

7. A mask device in accordance with claim 3, wherein fastening device has comprises, at the mask wall, an elastically deformable second passage sleeve formed jacketing around a part of the hose with an external thread and a nut with a conical internal thread for being screwed to the external thread of the second passage sleeve and thereby for fixing the hose in the holding position.

8. A mask device in accordance with claim 7, wherein an elastically deformable clamping sleeve (27) is formed jacketing around a part of the hose between a hose outer side and an inner circumferential surface of the second passage sleeve when viewed in a radial direction, wherein the material of the clamping sleeve has a higher strength than the material of the hose.

9. A mask device in accordance with claim 3, wherein the fastening device comprises an elastically deformable diaphragm suction sleeve arranged at the mask wall with a diaphragm inner volume and the diaphragm suction sleeve is formed jacketing in around the hose in an area of the passage opening, wherein the diaphragm suction sleeve has a pressure port for generating a vacuum in the diaphragm inner volume.

10. A mask device in accordance with claim 9, wherein an elastically and/or plastically deformable solid is arranged in the diaphragm inner volume.

11. A mask device in accordance with claim 3, wherein the fastening device comprises an elastically deformable diaphragm pressure sleeve with a diaphragm inner volume, which diaphragm pressure sleeve is arranged jacketing around the hose, in an area of the passage opening, wherein the diaphragm pressure sleeve has a pressure port for generating an overpressure in the diaphragm inner volume.

12. A mask device in accordance with claim 2, wherein the hose has a thinner hose wall in an area of the passage opening than in an area outside of the passage opening.

13. A mask device in accordance with claim 2 wherein the hose has an elastically deformable hose wall and/or a hose wall with a higher elasticity in an area of the passage opening than in an area outside of the passage opening.

14. A mask device in accordance with claim 3, wherein the fastening device comprises a thermoplastically deformable connection jacket, which is formed jacketing around the hose and is arranged at the mask wall in an area of the passage opening.

15. A mask device in accordance with claim 3, further comprising a connection hinge configured to articulatedly hold the hose in the passage opening.

16. A mask device in accordance with claim 2, wherein a part of the hose, which is located in a mask volume formed by the breathing mask at the patient in a use state of the mask device, is plastically deformable.

17. A mask device in accordance with claim 1, wherein the nasal cannula device has a gas discharge for discharging the ventilation gas into the nose of the patient, wherein the gas discharge has a nozzle configuration.

18. A mask device in accordance with claim 1, wherein the nasal cannula device has a gas discharge section for discharging the ventilation gas into the nose of the patient, wherein the gas discharge section tapers conically in a ventilation gas line passage direction.

19. A mask device in accordance with claim 1, wherein nasal cannula device has a gas discharge section for discharging the ventilation gas into the nose of the patient, wherein a balloon seal with a balloon jacket is formed in and/or at the gas discharge section, wherein the balloon jacket has a gas opening for feeding the ventilation gas in the nasal cannula device during a ventilation of the patient into a balloon volume formed by the balloon jacket, the balloon jacket being configured to provide a pressurized holding of the gas discharge section in the nose.

20. A mask device in accordance with claim 1, wherein the nasal cannula device has a gas discharge section for discharging the ventilation gas into the nose of the patient, wherein the mask device further comprises an inflatable hose seal, which is configured in a ring shape around the gas discharge section.

21. A mask device in accordance with claim 1, wherein the nasal cannula device has a gas discharge section for discharging the ventilation gas into the nose of the patient, wherein the gas discharge section has at least one elastically deformable spring-loaded arm configured to provide a pressurized holding of the gas discharge section in the nose.

22. A mask device in accordance with claim 1, further comprising a holding strap device with a headband and/or with a neckband formed at the breathing mask for holding the breathing mask at the patient.

23. A mask device in accordance with claim 1, wherein the breathing mask has a mask edge for placing the breathing mask over the face of the patient, wherein the mask edge has an elastically deformable silicone lip.

24. A mask device in accordance with claim 1, wherein the breathing mask has an elastically deformable mask edge for placing the breathing mask over the face of the patient, wherein the mask edge has an edge chamber with an edge volume, in which overpressure prevails relative to the area surrounding the mask device.

25. A mask device in accordance with claim 1, wherein the breathing mask has a gel and/or sponge mask edge for placing the breathing mask over the face of the patient.

26. A holding device for a mask device with a breathing mask configured to be positioned over the mouth and over the nose of a patient and a nasal cannula device for sending a ventilation gas into the nose of the patient, the holding device comprising:
means for holding the nasal cannula device at the breathing mask.

27. A mask device in accordance with claim 1, in combination with a gas source to form a ventilator.

28. A process for setting a mask device for a ventilator for ventilating a patient, the process comprising the steps of:
providing the mask device, wherein the mask device comprises: a breathing mask configured to be positioned over the mouth and over the nose of the patient; a nasal cannula device for sending a ventilation gas into the nose of the patient; and a holding device holding the nasal cannula device at the breathing mask, wherein the nasal cannula device has a hose for sending the ventilation gas and a gas discharge section for discharging the ventilation gas into the nose of the patient, and the holding device comprises a fastening device for fixing the hose in a holding position at the breathing mask as well as for releasing the hose WO from the holding position, for a displacement motion between the hose and the breathing mask;
positioning the breathing mask over the mouth and over the nose of the patient;
displacing the hose relative to the breathing mask for positioning the gas discharge section in or at the nose of the patient, and
fixing of the hose by the fastening device in the holding position.
